(19) United States
(12) Patent Application Publication
(10) Pub. No.: US 2002/0090360 A1
(43) Pub. Date: Jul. 11, 2002

(54) Inflammatory cytokine secretion inhibition

(76) Inventors: Anthony E. Bolton, Derbyshire (GB); Arkady Mandel, North York (CA); Daniel Nathan Sauder, Baltimore, MD (US)

Correspondence Address: Gerald F. Swiss, BURNS, DOANE, SWECKER & MATHIS, L.L.P., P.O. Box 1404, Alexandria, VA, 22313-1404, US

(21) Appl. No.: 10/002,634

(22) Filed: Dec. 5, 2001

(30) Foreign Application Priority Data
Dec. 5, 2000 (CA) 2,327,631
Dec. 5, 2000 (CA) 2,327,628

(51) Int. Cl. A61K 45/00; C12N 5/08

(52) U.S. Cl. 424/93.7; 435/372

(57) ABSTRACT

The present invention relates to a process of decreasing the expression of one or more of the inflammatory cytokines IFN-γ and IL-6 by cells in a mammalian patient, which comprises administering to the patient an effective amount of stressed mammalian blood cells, said stressed cells having been extracorporeally subjected to at least one stressor selected from oxidative stress and ultraviolet radiation. The process of the present invention can be used for alleviating chronic fatigue syndrome in a mammalian patient.

### CROSS-REFERENCE TO RELATED APPLICATIONS

This application claims convention priority from Canadian patent application serial number 2,327,631, filed Dec. 5, 2000, and from Canadian patent application serial number 2,327,628, filed Dec. 5, 2000, the disclosures of which are incorporated herein by reference in their entirety.

### FIELD OF THE INVENTION

This invention relates to treatment of biological cells and immune system modulation. More specifically, it relates to treatment of cells of the mammalian immune system to alter the cytokine profiles of certain types of constituent cells, and therapeutic applications of such treatments.

### BACKGROUND OF THE INVENTION

The mammalian immune system comprises lymphocytes (one type of white blood cell), the major components of which are B cells, which mature within the bone marrow, and T cells which migrate from the bone marrow to mature in the thymus gland. B cells react to antigens to proliferate and differentiate into memory B cells and effector B cells to generate and express antibodies specific to the antigen, thereby removing the antigen from the host. T cells have T cell receptors which recognize antigen associated with MHC molecules on a cell, and as a result of this recognition differentiate into memory T cells and various types of effector T cells. The T cell population is made up of T-helper (T_{H}) cells and T-cytotoxic (T_{C}) cells, distinguished from one another by the presence of the surface membrane glycoprotein CD4 on T_{H} cells and the surface membrane glycoprotein CD8 on T_{C} cells. Activation of a T_{H} cell can cause it to secrete various growth factors (cytokines). Different types of T_{H} cells secrete different cytokines. These cytokines play key roles in the immune response, including autoimmune responses.

One type of T_{H} cell, known as T_{H}1, expresses cytokines which, in excessive amounts, can cause inflammation in the mammalian body. Examples of such inflammatory cytokines include interferon-γ (IFN-γ), interleukin-6 (IL-6) and interleukin-12 (IL-12). When the body produces inappropriately large amounts of inflammatory cytokines, significantly more than endogenous levels found in the corresponding non-diseased tissue of healthy individuals, either through over-activation of T_{H}1 cells, activation of excessive numbers of T_{H}1 cells, or a switch of other types of T cells to the T_{H}1 type to create excessive numbers of cytokines expressing T_{H}1 cells, an inflammatory disorder can manifest itself in a patient.

### SUMMARY OF THE INVENTION

The present invention provides a process whereby expression of inflammatory cytokines including IFN-γ and IL-6, either individually or in combination, is reduced in a mammalian patient body. This process involves introducing blood cells into the patient which cells have been extracorporeally stressed by subjection to an oxidative stress and/or ultraviolet radiation. On introduction of these stressed blood cells, there is a reduction in the expression of one or more of these inflammatory cytokines, either by down regulating T_{H}1 cells, or perhaps by decreasing the population of T_{H}1 cells, e.g. by causing a switch of T cells from T_{H}1 to T_{H}2. Whatever the precise mechanism of action, the result is a significant and measurable decrease in these inflammatory cytokines in the patient's system. Accordingly, the process of the invention is useful in the medical treatment of patients suffering from, prone to, or at risk of contracting a disorder associated with excessive amounts of one or more of the inflammatory cytokines IFN-γ and IL-6 (e.g., chronic fatigue syndrome—see Cannon et.al., J. Clin. Immunol. 19(6): 414-21, 1999; and Gupta, S. et.al., Int. J. Mol.Med. 3(2): 209-13, 1999).

Thus according to the present invention, there is provided a process of decreasing the expression of one or more of the inflammatory cytokines IFN-γ and IL-6 by cells in a mammalian patient, which comprises administering to the patient an effective amount of stressed mammalian blood cells, said stressed cells having been extracorporeally subjected to at least one stressor selected from oxidative stress and ultraviolet radiation.

There is further provided a method for treating an inflammatory disease condition in a patient mediated by inflammatory cytokine production, which method comprises administering to the patient an effective amount of stressed mammalian blood cells wherein said stressed mammalian blood cells have been extracorporeally subjected to at least one stressor selected from oxidative conditions and ultraviolet radiation.

### BRIEF REFERENCE TO THE DRAWING

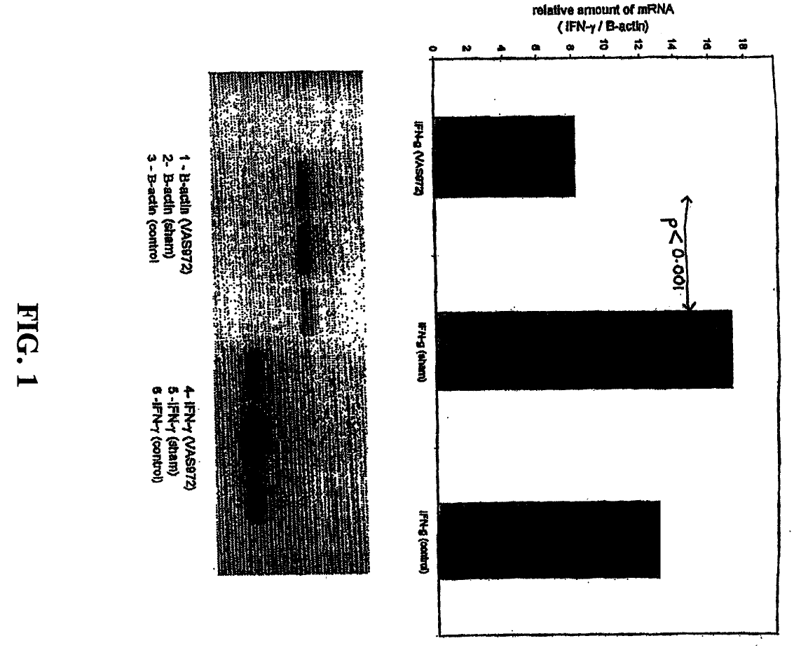

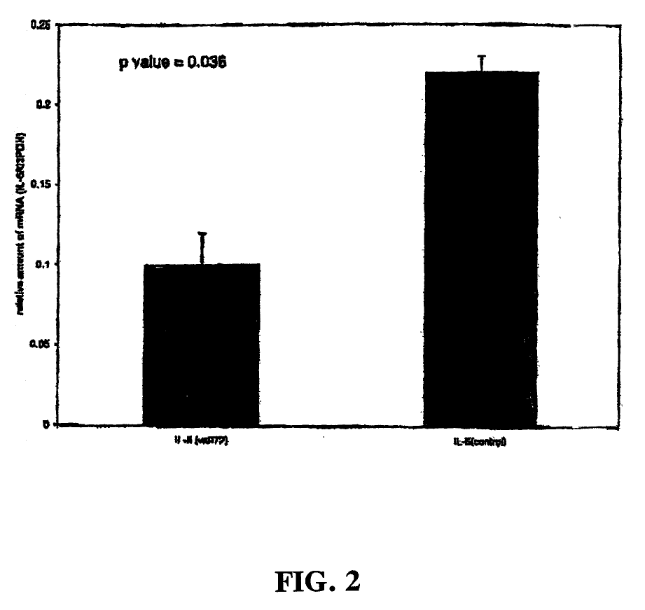

The accompanying FIGS. 1 and 2 are graphical presentations of the results of the experiments reported below in the Examples.

### DESCRIPTION OF THE PREFERRED EMBODIMENTS

A preferred embodiment of the invention is a process of decreasing the expression of IL-6 from cells in a mammalian patient, which comprises administering to the patient an effective amount of stressed mammalian blood cells, said stressed cells having been extracorporeally subjected to at least one stressor selected from oxidative stress and ultraviolet radiation. Such a process is useful in treating medical disorders associated with excess expression or excess presence of IL-6. A preferred application of the process of the present invention is in the treatment of disorders mediated by excess expression or excess presence of IL-6 other than graft versus host disease (GVHD); autoimmune diseases, such as rheumatoid arthritis, psoriasis, scleroderma, lupus, diabetes mellitus, organ rejection, miscarriage, multiple sclerosis, inflammatory bowel disease and atherosclerosis; and contact hypersensitivity disorders.

A particularly preferred embodiment is a process of decreasing IL-6 expression by cells in a mammalian patient which comprises administering to the patient an effective amount of stressed mammalian blood cells, said stressed cells having been extracorporeally subjected to at least one stressor selected from oxidative stress and ultraviolet radiation. This process is particularly useful for alleviating disorders such as chronic fatigue syndrome.

The source of the stressed blood cells for use in the present invention is preferably the patient's own blood, i.e. an aliquot of autologous blood.

The terms “aliquot”, “aliquot of blood” or similar terms used herein include whole blood; separated cellular fractions of the blood, including platelets; separated non-cellular fractions of the blood, including plasma; plasma components; and combinations thereof. Preferably, in human patients, the volume of the aliquot is up to about 400 ml, preferably from about 0.1 to about 100 ml, more preferably from about 1 to about 15 ml, even more preferably from about 8 to about 12 ml, and most preferably about 10 ml. The effect of the stressor or the combination of stressors is to modify the blood, and/or the cellular or non-cellular fractions thereof, contained in the aliquot. The modified aliquot is then re-introduced into the subject's body by any suitable method, most preferably intramuscular injection, but also including subcutaneous injection, intraperitoneal injection, intra-arterial injection, intravenous injection and oral administration, following which it causes decrease in the expression of one or more of the inflammatory cytokines INF-γ and IL-6.

According to a preferred process of the present invention, an aliquot of blood is extracted from a mammalian subject, preferably a human, and the aliquot of blood is treated ex vivo, simultaneously or sequentially, with the aforementioned stressors. Then it is injected back into the same subject. Preferably a combination of both of the aforementioned stressors is used.

Preferably, the aliquot of blood is in addition subjected to mechanical stress. Such mechanical stress includes stress that is that applied to the aliquot of blood by extraction of the blood aliquot through a conventional blood extraction needle, or a substantially equivalent mechanical stress, shortly before the other chosen stressors are applied to the blood aliquot. This mechanical stress may be supplemented by a mechanical stress exerted on the blood aliquot by bubbling gases through it, such as ozone/oxygen mixtures, as described below. Optionally, a temperature stressor may be applied to the blood aliquot, simultaneously or sequentially with the other stressors, i.e. a temperature at, above or below body temperature.

The optionally applied temperature stressor either warms the aliquot being treated to a temperature above normal body temperature or cools the aliquot below normal body temperature. The temperature is selected so that the temperature stressor does not cause excessive hemolysis in the blood contained in the aliquot, and so that, when the treated aliquot is injected into a subject, the desired effect will be achieved without the development of significant adverse side effects. Preferably, the temperature stressor is applied so that the temperature of all or a part of the aliquot is up to about 55° C., and more preferably in the range of from about −5°0 C. to about 55° C., but maintaining the aliquot largely in the liquid phase.

In some preferred embodiments of the invention, the temperature of the aliquot is raised above normal body temperature, such that the mean temperature of the aliquot does not exceed a temperature of about 55° C., more preferably from about 40° C. to about 50° C., even more preferably from about 40° C. to about 44° C., and most preferably about 42.5±1° C.

In other preferred embodiments, the aliquot is cooled below normal body temperature such that the mean temperature of the aliquot is within the range of from about 4° C. to about 36.5° C., more preferably from about 10° C. to about 30° C., and even more preferably from about 15° C. to about 25° C.

The oxidative environment stressor can be the application to the aliquot of solid, liquid or gaseous oxidizing agents. Preferably, it involves exposing the aliquot to a mixture of medical grade oxygen and ozone gas, most preferably by applying to the aliquot medical grade oxygen gas having ozone as a component therein. The ozone content of the gas stream and the flow rate of the gas stream are preferably selected such that the amount of ozone introduced to the blood aliquot, either on its own or in combination with one of the other stressors, does not give rise to excessive levels of cell damage, i.e. a degree of cell damage can be tolerated as long as it is without significant adverse side effects. Suitably, the gas stream has an ozone content of up to about 300 μg/ml, preferably 0.1 up to about 100 μg/ml, more preferably up to about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 litres/min, preferably up to about 0.5 litres/min, more preferably up to about 0.4 litres/min, even more preferably up to about 0.33 litres/min, and most preferably about 0.24±0.024 litres/min. The lower limit of the flow rate of the gas stream is preferably not lower than 0.01 litres/min, more preferably not lower than 0.1 litres/min, and even more preferably not lower than 0.2 litres/min, all rates at STP (0° C. and 1 atmosphere pressure). In the alternative, chemical oxidants such as hydrogen peroxide, permanganates and periodates, of biologically acceptable types and in biologically acceptable concentrations, can be used in the liquid phase to provide the required oxidative environment.

The ultraviolet light stressor is suitably applied by irradiating the aliquot under treatment from a source of UV light, i.e. electromagnetic radiation of wavelength from about 180-400 nm. Preferred UV sources are UV lamps emitting UV-C band wavelengths, i.e. wavelengths shorter than about 280 nm. Ultraviolet light corresponding to standard UV-A (i.e., wavelengths from about 315 to about 400 nm) and UV-B (i.e., wavelengths from about 280 to about 315 nm) sources can also be used. The UV dose should be selected, on its own or in combination with the other chosen stressor(s), so that excessive amounts of cell damage do not occur, and so that, when the treated aliquot is injected into a subject, the desired effect will be achieved. For example, an appropriate dosage of such UV light, applied simultaneously with the aforementioned temperature and oxidative environment stressor, can be obtained from lamps with a power output of from about 10 to about 30 watts, arranged to surround the sample container holding the aliquot, each lamp providing an intensity, at a distance of 16 mm, of from about 5 to about 20 mW/cm². Up to eight such lamps surrounding the sample bottle, with a combined output at 253.7 nm of 10-30 watts, operated at an intensity t deliver a total UV light energy at the surface of the blood of from about 0.025 to about 10 joules/cm², preferably from about 0.1 to about 3.0 joules/cm^{2,}, may advantageously be used. Such a treatment, applied in combination with the oxidative environment stressor, provides a modified blood aliquot which is ready for injection into the subject.

It is preferred to subject the aliquot to the oxidative environment stressor, the UV light stressor and the temperature stressor simultaneously, following the subjection of the aliquot to the mechanical stress, e.g. by extraction of the blood from the patient. Thus, the aliquot may be maintained at a predetermined temperature above or below body temperature while the oxygen/ozone gas mixture is applied thereto and while it is irradiated with ultraviolet light.

The time for which the aliquot is subjected to the stressors is normally within the time range of from about 0.5 minutes up to about 60 minutes. The time depends to some extent upon the chosen combination of stressors. When UV light is used, the intensity of the UV light may affect the preferred time. The chosen temperature level may also affect the preferred time. When oxidative environment in the form of a gaseous mixture of oxygen and ozone applied to the aliquot is chosen as one of the two stressors, the concentration of the oxidizing agent and the rate at which it is supplied to the aliquot may affect the preferred temperature. Some routine experimentation to establish optimum times may be necessary on the part of the operator, once the other stressor levels have been set, such experimentation being well within the skill of the art. Under most stressor conditions, preferred times will be in the approximate range of from about 2 to about 12 minutes, more preferably from about 2 to about 5 minutes, and most preferably about 3 minutes. The starting blood temperature, and the rate at which it can be warmed or cooled to a predetermined temperature, tends to vary from subject to subject. Warming is suitably by use of one or more infrared lamps placed adjacent to the aliquot container. Other methods of warming can also be adopted.

As noted, it is preferred to subject the aliquot of blood to a mechanical stressor, in addition to the chosen stressor(s) discussed above. Extraction of the blood aliquot from the patient through an injection needle constitutes the most convenient way of obtaining the aliquot for further extracorporeal treatment, and this extraction procedure imparts a suitable mechanical stress to the blood aliquot. The mechanical stressor may be supplemented by subsequent processing, for example the additional mechanical shear stress caused by bubbling as the oxidative stressor is applied.

In the practice of the preferred process of the present invention, the blood aliquot may be treated with the heat, UV light and oxidative environment stressors using an apparatus of the type described in aforementioned U.S. Pat. No. 4,968,483 to Müller et al. The aliquot is placed in a suitable, sterile container, which is fitted into the machine. A UV-permeable container is used and the UV lamps are switched on for a fixed period before the other stressors are applied, to allow the output of the UV lamps to stabilize. When a temperature stressor is also used, the UV lamps are typically on while the temperature of the aliquot is adjusted to the predetermined preferred value, e.g. 42.5±1° C. Four UV lamps are suitably used, placed around the container. The oxidative stressor is then applied.

In the preferred method of the invention, a mammalian patient under treatment for an IFN-γ mediated disorder or an IL-6 mediated disorder is given one or more courses of treatments, each course of treatment comprising the administration to a mammalian subject of one or more (e.g. one to six or one to twelve) aliquots of mammalian blood modified as discussed above.

For optimum effectiveness of the treatment, it is preferred that no more than one aliquot of modified blood be administered to the subject per day, in one or more injection sites, and that the maximum rest period between any two consecutive aliquots during the course of treatment be no greater than about 21 days. As used herein, the term “rest period” is defined as the number of days between consecutive aliquots or consecutive courses of treatment on which no aliquots of modified blood are administered to the subject.

Therefore, except where aliquots are administered to the subject on consecutive days, a rest period of from 1 to 21 days is provided between any two aliquots during the course of treatment. Moreover, at least one of the rest periods during the course of treatment preferably has a length of about 3 to 15 days.

Although it may be sufficient to administer only one course of treatment as described above to the subject, it may be preferred in some circumstances to administer more than one course of treatment, or to follow the above-described course of treatment by periodic “booster” treatments, if necessary, to maintain the desired effects of the present invention. For example, it may be preferred to administer booster treatments at intervals of 1 to 4 months following the initial course of treatment, or to administer a second course of treatments to the subject following a rest period of several weeks or months.

In view of the fact that the process of the invention described above leads to a significant decrease in the expression and/or activity of the inflammatory cytokine IL-6, the invention is particularly indicated for prophylaxis or alleviation of chronic fatigue syndrome (CFS) in human patients. Whilst the etiology of CFS remains contentious, there is a general consensus that IL-6 plays a role in CFS, either as a result of abnormal levels of IL-6 in the patient or abnormal sensitivity to IL-6 on the part of the patient. See, for example, Gupta S., et. al., J. Psychiatr. Res. 31(1): 149-156, 1997; Cannon J. G. et.al., J. Clin.Immunol. 19(6): 414-421, 1999; and Pall M. L., Med. Hypotheses 54(1): 115-25, 2000. Although excessive levels of and/or excessive sensitivity to IL-6 are almost certainly not the only factors controlling CFS in a pateient, they are at least a significant contributing factor, and the process and composition of the invention whereby IL-6 is downregulated accordingly shows potential in successful alleviation of this disorder.

The invention is further illustrated and described with reference to the following specific example, comprising animal studies conducted in an approved manner. The examples are offered for purposes of illustrating the invention and should not be construed as a limitation.

### EXAMPLE

As a measure of the effect of the process of the present invention on inflammation resulting from T cell secretions, a contact hypersensitivity (CHS) test was used, according to approved animal experimentation procedures, using the method described by Kondo et. al., Br J.Dermatol. 131:354-359, 1994, with minor variations. The disclosure thereof is incorporated herein by reference. Briefly, to induce CHS, the abdominal skin of each mouse was shaved and painted with dinitrodifluorobenzene (DNFB), the sensitizing chemical, using 25 μl of 0.5% DNFB in a 4:1 acetone:olive oil solution. This sensitization was applied to four groups of five Balb/c mice. In addition, a measure of the responsible cytokines was made.

Whole blood was obtained from Balb/c mice by cardiac puncture through an injection needle, and treated with an anti-coagulant. An aliquot of this anticoagulated blood was subjected to the process described herein, to obtain treated blood. The remainder was left untreated, for use in control experiments. Since the Balb/c mice used were genetically identical, the administration of the treated blood to others of the group is equivalent to administration of autologous blood.

To obtain treated blood, the selected aliquot, in a sterile, UV-transmissive container, was treated simultaneously with a gaseous oxygen/ozone mixture and ultraviolet light at elevated temperature using an apparatus as generally described in aforementioned U.S. Pat. No. 4,968,483 Müller et.al. Specifically, 12 ml of citrated blood was transferred to a sterile, low density polyethylene vessel (more specifically, a Vasogen VC7002 Blood Container) for ex vivo treatment with stressors according to the invention. Using an apparatus as generally described in the aforementioned Müller et al patent (more specifically, a Vasogen VC7001 apparatus), the blood was heated to 42.5±1° C. and at that temperature irradiated with UV light at a wavelength of 253.7 nm, while oxygen/ozone gas mixture was bubbled through the blood to provide the oxidative environment and to facilitate exposure of the blood to UV. The constitution of the gas mixture was 14.5±1.0 μg ozone/ml, with the remainder of the mixture comprising medical grade oxygen. The gas mixture was bubbled through the aliquot at a rate of 240±24 ml/min for a period of 3 minutes.

Of the 4 groups of sensitized mice, the first, control group A-1 received no treatment. The second, control group B-1, was treated with 50 μl of physiological saline. The third, control group C-1, was sham treated, with 50 μl of blood which had been extracted but not treated with the stressors. The fourth, test group D-1, was treated with 50 μl of blood subjected to stressors as described above. Treatments, each involving intramuscular injection of 50 μl of the respective liquid, started on the day of sensitization, and were repeated every day for a total of 6 days. On the same day as the last treatment, but after its administration, the animals were challenged with DNFB, by applying to one ear of each animal 10 μl of 0.2% solution of DNFB. Inflammation due to CHS manifests itself in a swelling of the ears. Ear thickness was measured 24 hours after challenge, with a Peacock spring-loaded micrometer (Ozaki Co., Tokyo, Japan). The results were expressed as the change (from pre-challenge level) in ear thickness and represent the mean maximal increase at 24 hours after challenge.

The animals were sacrificed, and lymph nodes draining the ear which was challenged with DNFB were collected. The expression of the mRNA for the cytokines IFN-γ and IL-6 was tested. This process of testing and analysis essentially followed the procedures described in Kondo, S., et.al., J.Immunology 4822:157, 1996. Thus the PCR products were determined by scanning of photonegatives using a laser densitometer, and the densitometric value of each was normalized to that of the housekeeping gene B-actin. The analyses indicated that animals which had received a course of injection of blood subjected to stressors as described had significantly reduced IFN-γ and IL-6 as compared with sham treated animals and controls, as illustrated in the accompanying Figures, in general correlation with the anti-inflammatory results.

Results shown in FIG. 2, a plot of relative amount of mRNA for IL-6 from the lymph tissue of the treated animals and the controls (saline treated), averaged across the animals of each group, are particularly noteworthy. The demonstrated reduction in IL-6 shows the potential of the process and compositions of the present invention in the prophylaxis or alleviation of chronic fatigue syndrome in human patients.

The results shown in FIG. 1, namely the reduction in the secretion of IFNγ from cells as a result of the process of the invention, suggest another therapeutic use of the present invention, namely in association with chemotherpeutic or radiation therapy of malignancies such as lymphomas. It is known that malignant cells in lymphomas such as chronic lymphocytic leukemia (CLL) secrete excessive amounts of IFN-γ, which then acts as protectant for the malignant cells against apoptosis inducing agents such as chemotherapeutic chemical agents and radiation treatments commonly adopted to treat the lymphomas. Inhibition of secretion of this cytokine from malignant cells accordingly renders them more susceptible to subsequent chemotherapeutic or radiation therapy. Another aspect of the present invention accordingly provides a process in which a mammalian patient undergoing or awaiting chemotherapy and/or radiation for a malignancy such as a lymphoma is given one or more courses of treatments of stressed mammalian blood cells as described above, to render the patient's malignancy more susceptible to chemotherapeutic or radiation therapy.

1. A method for treatment or prophylaxis of an inflammatory disease condition in a patient mediated by excess inflammatory cytokine production and/or abnormal sensitivity of the patient to one or more inflammatory cytokines, said cytokines being selected from the group consisting of IFN-γ and IL-6, which method comprises administering to the patient an effective amount of stressed mammalian blood cells wherein said stressed mammalian blood cells have been extracorporeally subjected to at least one stressor selected from oxidative conditions and ultraviolet radiation.

2. A method for treatment or prophylaxis of an inflammatory disease condition in a patient mediated by excess IL-6 production and/or abnormal sensitivity of the patient to IL-6, which method comprises administering to the patient an effective amount of stressed mammalian blood cells wherein said stressed mammalian blood cells have been extracorporeally subjected to at least one stressor selected from oxidative conditions and ultraviolet radiation.

3. The method of claim 2 wherein the disease condition is chronic fatigue syndrome.

4. The method of claim 3 wherein the stressed mammalian blood cells have been extracorporeally subjected to both oxidative conditions and ultraviolet radiation simultaneously.

5. The method of claim 4 wherein the stressed mammalian blood cells have additionally been extracorporeally subjected to heat stress simultaneously with subjection to both oxidative conditions and ultraviolet radiation.

6. The method of claim 5 wherein the oxidative conditions comprise bubbling a gaseous mixture of medical grade oxygen and ozone through the blood, for a period of from about 0.5 minutes to about 60 minutes.

7. The method of claim 6 wherein the gaseous mixture has an ozone content of from about 0.1 to about 100 μg/ml.

8. The method of claim 7 wherein the UV stressor is UV-C radiation.

9. The method of claim 8 wherein the temperature stressor is a temperature in the range from about 40 to about 55° C.

10. The method of claim 9 wherein the stressed mammalian blood cells comprise a volume of whole blood of from about 0.1 to about 400 mls.

11. A process of decreasing the expression of one or more of the inflammatory cytokines IFN-γ and IL-6 from cells in a mammalian patient, which comprises administering to the patient an effective amount of stressed mammalian blood cells, said stressed cells having been extracorporeally subjected to at least one stressor selected from oxidative stress and ultraviolet radiation.
